(19) United States
(12) Patent Application Publication
(10) Pub. No.: US 2014/0350406 A1
(43) Pub. Date: Nov. 27, 2014

(54) Dynamic Operation for Subarrays in Medical Diagnostic Ultrasound Imaging

(71) Applicant: Stephen W. Henderson, Menlo Park, CA (US)

(72) Inventor: Stephen W. Henderson, Menlo Park, CA (US)

(73) Assignee: Siemens Medical Solutions USA, Inc., Malvern, PA (US)

(21) Appl. No.: 13/902,237

(22) Filed: May 24, 2013

(51) Int. Cl. A61B 8/00; B06B 1/02; B06B 1/06

(52) U.S. Cl. CPC A61B 8/4494; B06B 1/0607; A61B 8/4488; B06B 1/0215
USPC 600/459

(57) ABSTRACT

Dynamic operation is provided in sub-array ultrasound imaging. Rather than using fixed or Euclidian distance relative timing, an approximation determines the relative timing. The dynamic timing relationship is approximated with log(x), arctan(x), or 1/x relationships. Adders, registers, counters, comparators, or other simple circuitry is used to calculate the timing relationship. For example, a portion of the circuit featuring a digital comparator and counter produces a reciprocal relationship.

### BACKGROUND

The present invention relates to dynamic focusing in sub-arrays. In particular, relative delay or phase between elements for sub-array beamformation is provided.

Medical diagnostic ultrasound imaging systems have a limited number of receive beamformer channels. To maximize the number of elements used, partial beamforming combines signals from multiple elements for processing by a single receive beamformer channel. Signals from different elements are summed together to form a partially beamformed sub-array signal. The subarray signal is responsive to each of the plurality of elements and may be processed with a single receive beamformer channel.

To focus the sub-array, relative phasing or delays between elements is applied prior to summation. For example, the relative phase relationships are slowly varying for elements in close proximity or in a same sub-array. The phase traversed over depth is not very large, such as a few wavelengths. The relative phase for dynamic focusing is calculated using Euclidian distances. Such calculations include a square root and multiplication functions, so are computationally expensive, especially for multi-dimensional arrays with lots of elements for which sub-arrays are typically used. To avoid these problems, a fixed rather than dynamic phase relationship may be used between elements of a sub-array. The same relative phase is applied regardless of depth. For larger sub-arrays, the fixed phasing may result in less focus or resolution.

### BRIEF SUMMARY

By way of introduction, the preferred embodiments described below include systems, circuits, and methods for dynamic operation in sub-array ultrasound imaging. Rather than using fixed or Euclidian distance relative timing, an approximation determines the relative timing. The dynamic timing relationship is approximated with log(x), arctan(x), 1/x, or other relationships. Adders, registers, counters, comparators, or other simple circuitry is used to calculate the timing relationship since cordic functions or multiplication are not needed for the approximation. For example, a portion of the circuit featuring a digital comparator and counter produces a reciprocal relationship.

In a first aspect, a circuit is provided for dynamic operation in a sub-array of ultrasound imaging. A first adder and a first register are operable to generate a second order polynomial as a function of depth. A counter and a comparator are operable to generate signals representing a reciprocal of the second order polynomial. A second adder is operable to generate a relative timing for an element from the reciprocal. A sub-array beamformer is operable to delay or phase ultrasound signals for the element based on the relative timing.

In a second aspect, a method is provided for dynamic phase or delay calculation in a sub-array of ultrasound imaging. Dynamic timing relationships between elements of a sub-array are determined with a log(x), arctan(x), or 1/x function, where x is time. Signals from the elements of the sub-array are beamformed as a function of the dynamic timing relationships.

In a third aspect, a circuit is provided for dynamic operation in a sub-array of ultrasound imaging. A comparator is operable to produce a reciprocal of a derivative. A sub-array beamformer is operable to delay or phase ultrasound signals for an element based on the reciprocal of the derivative.

The present invention is defined by the following claims, and nothing in this section should be taken as a limitation on those claims. The various aspects described above may be used individually or in any possible combination. Other aspects and advantages are discussed below in conjunction with the preferred embodiments. These further aspects and advantages may be used independently of any of the aspects described above.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a system for sub-array beamforming;

FIG. 2 illustrates example ideal relative phase/delay curves offset to all start at zero;

FIG. 3 illustrates a first difference of the ideal phase curves of FIG. 2;

FIG. 4 illustrates a reciprocal of the first differences of FIG. 3;

FIG. 5 illustrates a difference of the reciprocals of FIG. 4;

FIG. 6 shows one embodiment of a circuit for generating relative timing for an element of a sub-array;

FIG. 7 illustrates an example second order polynomial;

FIG. 8 illustrates an example impulse chart representing a reciprocal of the second order polynomial of FIG. 7;

FIG. 9 illustrates an example relative phase accumulated from the impulses of FIG. 8; and

FIG. 10 shows one embodiment of a method for generating relative timing for an element of a sub-array and sub-array beamforming.

### DETAILED DESCRIPTION OF THE DRAWINGS AND PRESENTLY PREFERRED EMBODIMENTS

A simple circuit for generating the dynamic relative phase or delay (i.e., timing) profile between nearby elements is provided. For example, a few (e.g., 2-4) adders, one comparator, one counter, and a few storage registers (e.g., 4-5) are used for each element in sub-array focusing.

The circuit approximates the dynamic timing relationship over depth in a small sub-array with simple log(x), arctan(x), 1/x, or similar functions with negative second derivatives rather than calculating the ideal timing using Euclidian distance. A sub-circuit featuring a comparator may produce a reciprocal relationship to generate log(x), 1/x or similar approximations with low complexity, gate count, and/or clock rates. The registers of the circuit store static or initial values for approximating the desired timing relationship. Offline processes compute these circuit parameters for a line of focus.

The number of elements per sub-array may be increased as compared to sub-arrays applying static phase focusing given a fixed focal error. Since static timing may limit the size of the sub-arrays, larger sub-arrays may be used due to the timing approximation. As compared to static timing, better focusing and sensitivity may be provided.

The circuit may operate with slow clock rates, no multipliers or dividers, and/or no cordic operations. Memory, such as look-up table, and corresponding memory access, such as RAM access, is limited since a complete table of ideal timing relationships is not stored. There is low gate and bit count due to the minimal configuration information per channel. The circuit operates independently after a line is set-up by loading the registers. There is no accesses or loading of timing during a receive phase.

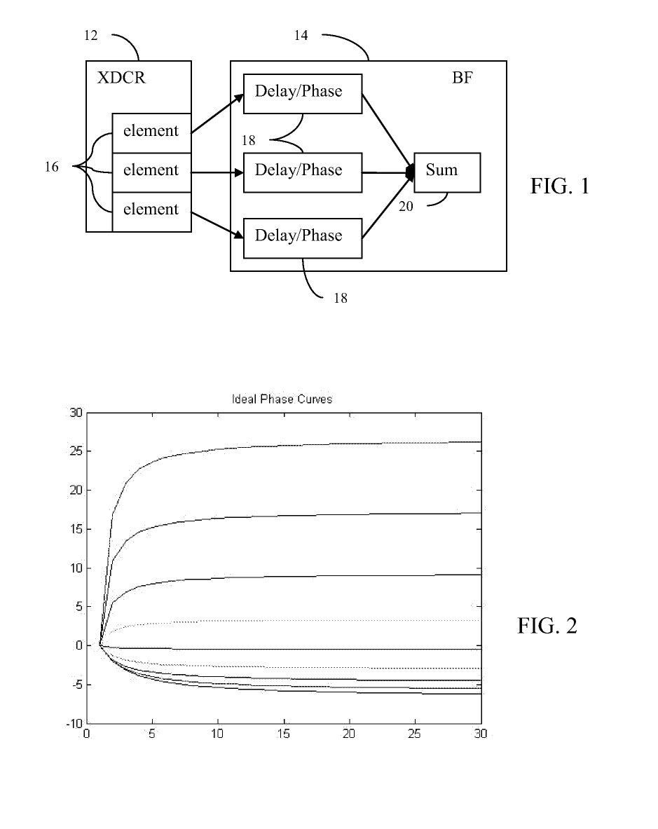

FIG. 1 shows a system for ultrasound beamforming. The system is a transducer 12 connected with a receive beamformer 14. The connection is fixed or releasable. For example, the transducer 12 and beamformer 14 are part of a medical diagnostic ultrasound imaging system. A cart, handheld, wearable, portable, briefcase, or other type of ultrasound imaging system is used. Additional, different, or fewer components may be provided. For example, detectors, scan converters and/or filters for generating an image from the beamformed data are provided.

The transducer 12 is an array of elements 16 in a housing. The housing is a handheld probe housing. In other embodiments, the housing is cardiac catheter, indo-cavity probe, intra-operative probe, or other transducer housing.

The transducer 12 is fixedly attached or releasably attached to the beamformer 14. In one embodiment, the beamformer 14 is a sub-array beamformer. The beamformer 14 operates on a fixed sub-set or a selectable sub-set of the elements 16. Any size sub-array may be used, such as 32×32, 16×16, 4×4, 16×4, 8×8, 8×1, 4×3, or other arrangement. The beamformer 14 is adjacent to the transducer 12. The beamformer 14 is within the probe housing, but may be in other positions, such as in an imaging system.

Outputs of the beamformer 14 are provided to another beamformer for forming beamformed samples from sub-array samples. A cable or coaxial cables provide the sub-array beamformed samples to a beamformer of the imaging system. Alternatively, the beamformer 14 beamforms across the entire array rather than a sub-array.

The elements 16 are piezoelectric or capacitive microelectromechanical ultrasound transducer (CMUT) elements. The elements 16 are distributed in a fully sampled multi-dimensional grid as a multi-dimensional transducer array. One-dimensional, 1.5D, sparse sampling or other grid spacings of elements may be provided. Any now known or later developed array of elements may be used. Any number of elements 16 may be used, such as 164, 356, or thousands. The array is divided or divisible by selection into any number of sub-arrays. Each sub-array is a group of elements, such as adjacent elements. The signals for elements in a sub-array are combined by beamformation to reduce the number of cables needed to transmit the received signals to the imaging system.

The beamformer 14 is provided for each sub-array. A different or same beamformer 14 is used for each sub-array. The number of sub-arrays matches a number of beamformer channels in a system side beamformer. The number of sub-arrays and corresponding number of sub-array beamformers 14 corresponds to the number of available cables connecting the transducer 12 to the imaging system and/or the number of receive beamformer channels in the diagnostic medical imaging system.

The sub-array beamformer 14 includes timing devices 18 and a summer 20. Additional, different, or fewer components may be provided. For example, an analog-to-digital converter is provided. As another example, filters, buffers, or other receive beamforming components are provided. In yet another example, a switching matrix or multiplexer selectably connects different elements 16 to different timing devices 18.

The timing devices 18 are any now known or later developed device for selecting signals based on travel time of acoustic energy. Analog or digital delays, such as shift registers, may be used. A phase rotator or shifter may be used. The phase shifter applies a phase shift of in-phase and quadrature or radio frequency signals to rotate a phase of the ultrasound signals by the relative timing. The signals from different elements 16 are relatively delayed and/or phased to focus the acoustic energy. The timing, either or both of delaying and phasing, is along the azimuth, elevation, or both azimuth and elevation directions. Different or the same amount of phase shift, depending on the distance from the corresponding elements to the focal point, is applied. Since different elements 16 of the sub-array are different distances away from a given depth along a scan line, different amounts of delay or phasing are applied to identify the signals from the depth on the line. As signals are received from different depths over time, different amounts of delay and/or phasing are applied to focus signals from a given element.

The summer 20 is a single summer, but may be part of a cascade of summers for the sub-array. The summer 20 is an analog summer and combines the relatively timed signals from the different elements 16 of the sub-array. In one embodiment, the summer comprises an operational amplifier. One input of the amplifier is grounded and the other input of the amplifier connects to different element channels or timing devices 18. The operational amplifier converts the current output by each path to a combined voltage signal. The virtual ground summation node of the operational amplifier provides isolation between the channels and avoids problems with parasitic capacity loading at the summation node, so a large number of paths may be summed without limiting the circuit bandwidth.

The output of the summer 20 is an output sub-aperture signal. Other summers 20 output sub-aperture signals for other sub-arrays. The sub-aperture signal is further beamformed by relative delaying and/or phasing and summing with other sub-aperture signals.

FIG. 2 shows some representative relative phase curves between elements in a subarray, and translated such that the curves all start at zero so as to emphasize their comparative shapes rather than absolute values. The x-axis is time or depth. Signals from different depths take different amounts of time to reach the elements 16, so time and depth may be used interchangeably. The y-axis gives the phase in degrees. These curves are ideal, such as calculated using Euclidian distance (i.e., square root of a sum of squares) from the elements to the focal points at different depths along a scan line. The scan line represented in FIG. 2 is not orthogonal to the sub-array, such as being steered at 10 degrees away from orthogonal in azimuth. Other numbers of elements, scan line positions, and/or phase curves may be used. In alternative embodiments, the curves represent delay curves. The examples below use phase, but the same embodiments may use delays or a combination of delays and phasing.

To avoid the relatively complex calculations to derive the phase curves of FIG. 2, an approximation is used. For example, the curves are approximated as log(x), 1/x, or arctan(x) function, where x is depth. A curve approximating k₀, +k₁/x may be used, where k is a constant or is part of a sequence with values that vary by depth. Other functions may be used to approximate timing. The approximation provides a similar, even if not ideal, curve shape, where a greater change in relative timing occurs at close depths and smaller change occurs for farther depths.

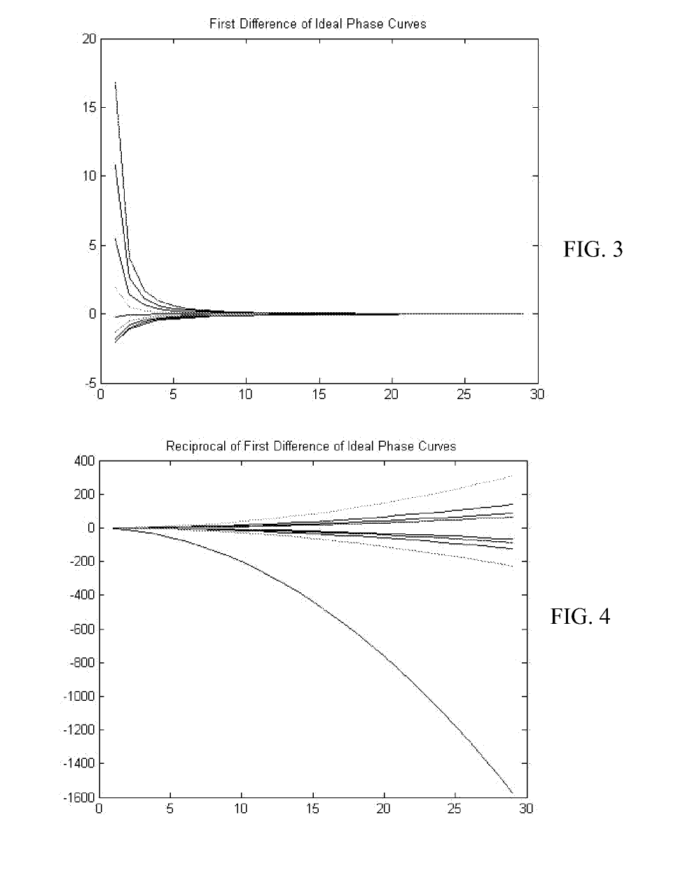

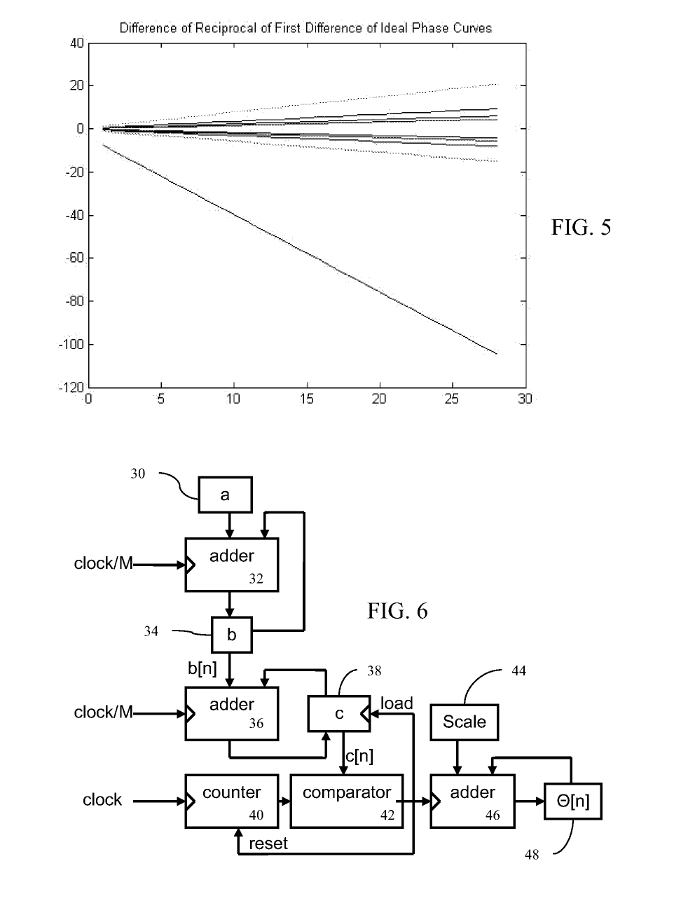

To characterize the ideal phase curves for approximation, the curves are converted to find parameters. In one approach, a first derivative of the curves is used. The change in depth is a 1/x function such as −1/x². FIG. 3 shows the first derivative as a difference between the curves. The approximation includes a reciprocal function, so the reciprocal of the first difference is determined, as shown in FIG. 4. The reciprocal is −x². This function still represents a multiplication. The change in depth d/dx of −x² is given by −2x. This is a straight line. FIG. 5 shows lines determined by a difference operation on the reciprocal. In inverse, the linear slope is generated, integrated, reciprocated, and then integrated to produce the desired approximation curve.

By determining timing based on characteristics of the relative timing as represented by the linear slopes and corresponding lines, the approximation characteristics to be implemented by a circuit are determined. For example, accumulation or integration over depth or time provides a straight line. By scaling the accumulation and setting the inputs to be accumulated, the desired curve is produced. The curve over depth is an integral of x. For a higher order, the integration is of the form kx-c. The line characteristics are used to provide the desired curve shape that is then scaled and offset at a final stage to desired phase values.

FIG. 6 shows one embodiment of a circuit for generating the timing. The relative timing for a given element is generated dynamically. The timing changes over depth so that the focus changes over time for a given receive line scan. By changing the relative timing over depth, better focusing than using static phase difference is provided. For a given phase error tolerance, more elements may be used in a sub-array as compared to using the static phase.

The amount of timing shift (e.g., amount of delay or phase shift) is relative to a center element of the sub-aperture, but may be relative to other elements in the sub-array.

The circuit is provided for each element or input channel of the sub-array beamformer 14. Since there may be hundreds or thousands of elements, the circuit is kept simple to avoid generating heat by the transducer 12 and to use less space. Based on clock inputs and loaded register values, the circuit generates relative timing information used by the timing devices 18 of the sub-array beamformer 14. The circuit operates on unsigned values until the scaling in a final stage, but may operate with signed values in other embodiments.

The circuit of FIG. 6 includes five registers 30, 34, 38, 44, 48, three adders 32, 36, 46, one comparator 42 with a latching stage, and one counter 40. Additional, different, or fewer components may be provided. For example, the register 30 and adder 32 are not provided. Rather than generating a second order polynomial, a first order polynomial is created. As another example, additional components to weight, scale, or otherwise alter the output, intermediary calculations, or inputs are provided.

The circuit does not include a multiplier, a divider, nor a device for performing a cordic operation. Instead, the circuit implements a polynomial function as a derivative and takes a reciprocal of the derivative. A memory or look-up table may be used to preload the registers for dynamic focusing along a line, but no memory or RAM access is needed during the dynamic focusing or during the calculation of the relative timing. Since the registers are loaded with characteristics for modeling an approximation, lower bit widths or memory sizes are needed as compared to a table of relative phases.

The circuit is implemented as discrete digital components. In other embodiments, an application specific integrated circuit (ASIC) includes or implements the components. A circuit of transistors and other devices in an ASIC may provide the functions without the component separation. In an alternative embodiment, a digital signal or other processor (e.g., field programmable gate array) implements the circuit.

The circuit operates in response to clock signals. The counter 40 operates based on the system or other clock, but may operate at a slower rate. The adders 30, 34 operate at a fraction of the clock signal. Any fractional value, represented by M in FIG. 6, may be used. For example, M=256. At a clock rate of 20 MHz, the adders 30, 34 are operated at a rate of just 100 kHz. Other rates of the clock or the fraction of the clock may be used. The ratio of the clock to M sets a sampling density for focusing along the scan line.

The adders 32, 36, and 46 are multiple bit adders. For example, the adders add two multi bit values together. Any now known or later developed adder may be used. Each adder 32, 36, and 46 is of the same type, but may be different types of adders. With a feed back of the output of the adder through a register 34, 38, or 48, the adder 32, 36, 46 operates as an accumulator or integrator. In an alternative embodiment, adders 32, 36 running at the divided clock rate may be a single adder shared using a time-multiplexing scheme.

The registers 30, 34, 38, and 48 are memories, buffers, or other data storage. Any memory for storing a multi-bit value may be used. Any now known or later developed register may be used. The registers 30, 34, 38, and 48 are each the same type or may be of different types of registers.

The registers 30, 34, 38, and 48 store respective single values (e.g., multi-bit word), but may store multiple values, such as in a first in first out configuration for register 30. For a given receive scan line, the registers 30, 34, 38, and 48 are loaded with initial values. Different scan lines result in the same or different values. Registers for different elements and a same scan line may have the same or different values. The values depend on the position of the element relative to the scan line and a reference phase or element.

The values initially loaded into the registers 30, 34, 38, and 48 are the same or different. Register 30 may be loaded with a constant value for a given line. The value does not change over depth or time. Other registers 34, 38, and 48 are loaded with initial values. The initial values are replaced by sums determined by respective adders. For increasing depths, the values in the registers 34, 38, and 48 change. One or more depths may be associated with the same values.

The counter 40 is a shift register, adder, multiplexer, memory, combinations thereof and/or other component for incrementing a count in response to input clock signals. Any now known or later developed counter may be used.

The comparator 42 is a digital comparator that generates a non-zero output when the value of counter 40 is equal to the value c[n] maintained in register 38. The comparator 40 may, alternatively, generate a non-zero output when c[n] is greater than the counter value. Other comparators may be used.

Overall, the circuit creates a sequence of signals over time or depth approximating the ideal phase curve. The sequence at the output is of relative timing representing 1/x, log(x), arctan(x) or similar functions.

The circuit is conceptually divided into three portions. In a first portion, a polynomial is created. The polynomial is created using the divided clock rate. The registers 30, 34, and 38 and the adders 32 and 36 generate a sequence of values as a function of depth. The sequence represents a second order polynomial. Alternatively, a sequence representing a first order polynomial is created using one fewer register and one fewer adder. The polynomial is created as a function of depth.

By incrementing the clock, the adders 32 and 36 are triggered to add a next value for a next depth. The registers 30, 34, and 38 provide the values to be added. For example, the adder 32 adds the constant value from the register 30 with the initial value or previous output of the adder 32 value from the register 34. The result is stored in the register 34. The result replaces the previous value or the initial value in the register 34. The values of the register 34 are a first order polynomial.

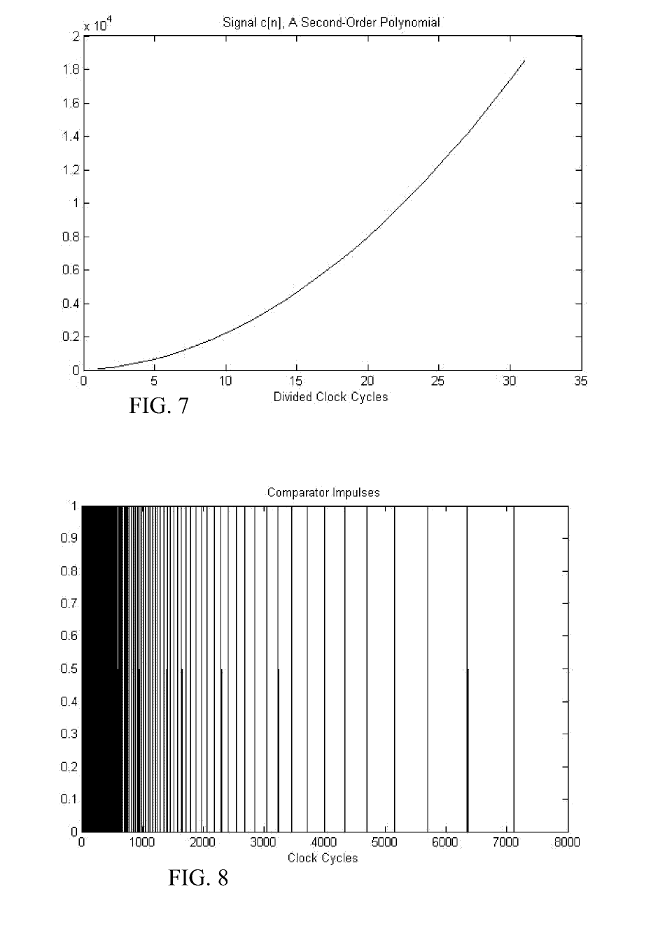

To create a second order polynomial, the adder 36 adds the value from the register 34 with the value from the register 38. The output from the adder 36 is stored in the register 38, replacing the initial value or a previous value loaded into the register 38. The values of the register 38 are the second order polynomial. At a given depth, as represented by the divided clock inputs, a value of the second order polynomial is provided. A sequence of such values is the second order polynomial as a function of depth. As represented in FIG. 6, this portion of the circuit calculates c[n]=(a/2)n²+(b₀+a/2)n+c₀ where n is depth, a is a constant value, b₀ is an initial value, and c₀ is another initial value. FIG. 7 shows an example second order polynomial for an element.

Another portion of the circuit creates a reciprocal of the second order polynomial. The counter 40 and the comparator 42 generate impulses at a rate proportional to the reciprocal of the second order polynomial. The counter 40 counts clock cycles or divided clock cycles. The comparator 42 compares the clock cycle count from the counter 40 to the second order polynomial c[n] from the register 38. The second order polynomial changes values once every M clock cycles. For lower valued portions of the polynomial curve c[n], the count will more frequently exceed the polynomial during a given depth cycle, resulting in a more rapidly changing phase/delay result. Conversely, as c[n] approaches a large value, the count less frequently exceeds c[n], making the delay/phase result change slowly and thus converge on a final value.

The output of the comparator 42 is pulses. A pulse is generated each time the count exceeds the current value of the polynomial output by the register 38. The result is pulses frequency modulated as a function of the depth where a greater number of pulses are generated over time for shallower depths. Each pulse resets the counter and causes loading of a current value of the register 38 for comparison. The value in the register 38 is the same value previously used until a new divided clock cycle increments the polynomial value. The pulse train represents a reciprocal of the derivative or second order polynomial (e.g., 1/c[n]). The circuit converges to a stable desired value at increasing depth due to the nature of ever increasing impulse intervals produced by the comparator 42. FIG. 8 shows an example pulse train over depth.

The remaining portion of the circuit generates the relative timing, such as phase, delay, or phase and delay as a function of depth. The adder 46 and registers 44 and 48 generate the timing based on the output of the comparator 42. The pulses trigger the adder 46 to add the scale value from the register 44 with the timing value from register 48. Where multiple pulses from the comparator 42 occur in one sample period (e.g., clock rate divided by M), then multiple sums occur to determine the timing value for the scan line sample. The resulting sum from the adder 46 is stored in the register 48.

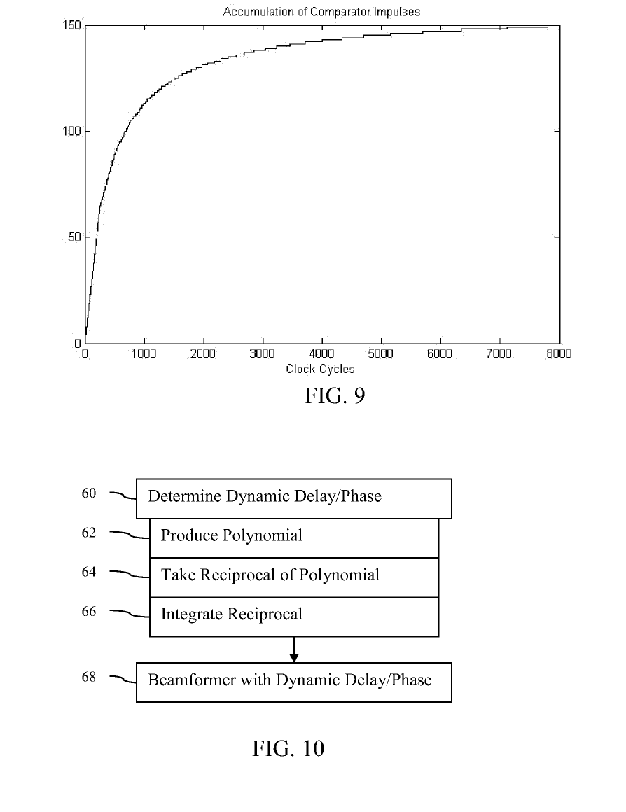

The adder 46 accumulates or integrates based on the pulses from the comparator 42, converting the pulses into the desired phase or delay curve. FIG. 9 shows an example phase curve of the timing values in the register 48 over time for a scan line. The relative timing is created using the reciprocal. The adder 46 integrates a signal proportional to 1/c[n] with a configurable scaling and offset.

The scale value is a constant. The constant is the same for the entire line, but different constants may be provided for different lines. The timing value stored in register 48 has an initial offset value that is replaced over time or depth with accumulated values.

The values a, b, c and θ for the registers 30, 34, 38, and 48 are established using calculation or experimentation. For example, let the initial values of a, b, c, and θ for the registers 30, 34, 38, and 48 are denoted a₀, b₀, c₀, and θ₀. The parameters a₀, b₀, c₀, Scale, and θ₀ for a given sub-array element and straight-line progression of receive focusing may be determined through a computational process. The computational process is executed on a microprocessor, FPGA, or other device outside of the circuit itself, and the resulting parameters are loaded into the circuit registers 30, 34, 38, and 48 prior to the receive phase of the ultrasound acquisition. First, the process computes ideal relative delay/phase curves over depth by evaluating differences in path lengths at each consecutive point of focus using well known Euclidean distance expressions (i.e., square-root of sum-of-squares of differences in Cartesian coordinates over the path of receive focus). Let this ideal relative delay/phase curve as a function of discrete depths n be denoted θ_{ideal}[n]. From θ_{ideal}[n] the process computes θ_{ideal}[n]=1/(θ_{ideal}[n₊1]−θ_{ideal}[n]). The process sets c₀=θ_{ideal}[n] so that the circuit starts with the same value for its computed c[n] as the ideal curve. Let N correspond to the maximum computed focus depth. From the circuit, it can be shown that c[N]=c₀+(N)b₀+N(N+1)a₀/2. The process sets c[N]=θ_{ideal}[N] so that the circuits computed c[n] will converge to the same value as the corresponding ideal curve. The process then sets b₀=θ_{ideal}[1]−θ_{ideal}[0] so that the initial slope (1^{st}-difference) of c[n] matches that of c_{ideal}[n]. The process then solves for a₀, which evaluates to: 2(c[N]−c₀−(b₀)N)/(N(N+1)). The process then emulates the circuit behavior over depth with provisional parameterization of Scaleₚᵣₒᵥᵢₛᵢₒₙₐₗ=1 and θₚᵣₒᵥᵢₛᵢₒₙₐₗ=0 and previously computed a₀, b₀, c₀ values. The process thus generates outputs θ_{emulated} [N]. The process then determines non-provisional value of Scale (register 44) as (θ_{ideal}[N]−θ_{ideal}[0])/(θ_{emulated}[N]−θ_{emulated}[0]). Lastly, the process determines the non-provisional value of θ₀ as: θ_{ideal} [N]−Scale×θ_{emulated}[N]. Other curve fitting approaches for deriving polynomial coefficients a₀, b₀, and c₀ may be possible.

Offline computations are performed to predetermine the register values, constants and/or initial values. A field programmable gate array, look-up table, or other device provides the register values prior to dynamically receiving along the scan line. For example, the values are loaded during a period of delay for reverberation and/or during transmit operations. Alternatively, real-time or as needed computation is used.

In other embodiments, different approximations are made. The circuit may be altered or replaced with other portions and/or components. Other reciprocal circuits, accumulation circuits, and/or polynomial generating circuits may be used. Other representations than pulses or curves over time may be used.

FIG. 10 shows a method for dynamic phase or delay calculation in a sub-array of ultrasound imaging. The method is implemented by the system of FIG. 1, the circuit of FIG. 6, or other systems and/or circuits. A processor, application specific integrated circuit, field programmable gate array, digital circuit, analog circuit, or combinations thereof is designed, configured, or instructed to perform the acts of FIG. 10.

The acts are performed in the order shown or a different order. Additional, different, or fewer acts may be provided. For example, acts 62, 64, and 66 relate to one approach for approximating the ideal phase curve without multiplication or cordic functions. Other acts for other approximations may be used. As another example, act 68 is not performed. In yet another example, acts for further beamformation from beamformed sub-array samples and image processing from the further beamformation output are provided. An image is generated using the sub-array signals for which dynamic focus is provided through approximation of the ideal delay or phase curve.

In act 60, the dynamic timing relationship is determined. The act is performed for all or some elements in a sub-array. Similarly, the act is performed for each of a plurality of sub-arrays. Depending on the location of the element relative to the focal position, different delay and/or phasing are needed. Since adjacent elements or elements in a contiguous sub-array have more similar phasing or delay than elements spaced from the sub-array, the relative timing is determined. The sub-array beamforming for each sub-array is concerned with relative timing amongst the elements of the sub-array. To beamform for the sub-array, the signals from the different elements are relatively timed based on the dynamic timing relationship.

Dynamic focus is provided. For a given scan line, the focus changes over time. Initially received signals are from closer depths than later received signals. By varying the focus over time, beamformed signals for different depths are provided.

Rather than rely on an ideal timing curve or static (single) focus, the dynamic timing relationships for the elements of a sub-array are determined with a log(x), arctan(x), 1/x or similar function, where x is time. An approximation is used to determine the phase, delay, or phase and delay for sub-array beamforming.

Acts 62, 64, and 66 represent one example approach for approximating the timing curve. In act 62, a second order polynomial is generated. The polynomial provides values as a function of the time. For different sample locations along the scan line, a different value is provided. A same value may be provided for some sample locations. The values follow the polynomial curve.

Any approach for generating the values along a polynomial curve may be used. For example, a constant value is added with a previous result of the adding for each of multiple increments in time. The result is used as the previous result in a next increment. The result is also used for another addition. This other addition adds the result with another previous result from this other addition. This other result is used for feed back to the other addition as well as represents the polynomial curve as a derivative.

In act 64, a reciprocal of the second order polynomial is determined. The reciprocal is over time. Any approach may be used. In one embodiment, the reciprocal is produced with a comparator. By comparing the derivative with a counter, pulses modulated in frequency to represent the reciprocal are formed. Other representations may be used, such as a curve or series of values.

In act 66, the dynamic timing relationship is created. The reciprocal is integrated and scaled to provide the timing for an element. In one embodiment, the scale is added to a previous timing relationship (e.g., added to a phase or delay). The addition occurs when triggered by the pulses of the reciprocal. The number of pulses in a given sampling period determines the amount of integration and resulting timing for that sampling period.

The timing relationship over time is the approximation of the phase or delay curve for the element. Acts 62, 64, and 66 are performed for other elements. Depending on the position of the element relative to the focal location, different relative timing is generated. Using different initial and/or constant values, a different relative timing over depth is determined for the different elements.

In act 68, the signals from elements of the sub-array are beamformed. The signals from the different elements are relatively delayed, phased, or delayed and phased. Using the timing relationship, the timing of the signals is adjusted. The relative timing across the elements of the sub-array focuses the received signals. The signals received from a focal location at a given depth are determined or selected by application of the timing. The focal location changes over time, so different timing is used to find the signals for the focal location at different times.

Azimuth, elevation, or azimuth and elevation electronic focusing is provided. By applying timing, electronic focusing is used. Mechanical focus may be used for one direction, such as elevation.

The relatively delayed or phased signals from the different elements are combined. By summing the signals, sub-array beamformed signals representing acoustic echoes from a focal point that varies over time are provided. The beamforming for this dynamic receive operation of the sub-arrays is performed using the dynamic timing relationships.

Using the circuit described above in FIG. 6 or the method of FIG. 10, the approximation of the ideal phase curve (phase over depth) may introduce some error as compared to the ideal phase curve itself. For example, in a scan line steered at 45 degrees from orthogonal to the sub-array, the maximum absolute phase error is 0.0408 wavelengths. The mean phase error is 0.0128 wavelengths. In another example, in a scan line steered at the orthogonal (i.e., 0 degrees), the maximum absolute phase error is 0.0859 wavelengths, and the mean phase error is 0.0184 wavelengths. These errors are better than using static focusing. Performance may be better, particularly in shallow regions, as compared to static focusing.

Since the ideal phase curve is not used, complex circuitry is avoided. Rather than multipliers or performing cordic functions, simpler circuitry is used. With numerical scaling used in simulation, each channel may be implemented with about 500 gates. 46 bits of configuration may used per channel. The parameter data may be compressed to exploit correlation with adjacent channels. For example, some of the same register values are used in adjacent elements. Other numbers of gates, bit amounts, bit sizes of register values, or combinations thereof may be used.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A circuit for dynamic operation in a sub-array of ultrasound imaging, the circuit comprising:
a first adder and first register to generate a second order polynomial as a function of depth;
a counter and a comparator to generate signals representing a reciprocal of the second order polynomial;
a second adder to generate a relative timing for an element from the reciprocal; and
a sub-array beamformer to delay or phase ultrasound signals for the element based on the relative timing.

2. The circuit of claim 1 wherein the second order polynomial is generated by the first adder and a third adder, the first adder adding a first value from the first register and a second value from a second register and outputting a first sum to the second register, the third adder adding the first sum from the second register and a third value from a third register and outputting a second sum to the second register, the second sum comprising the second order polynomial at a first depth.

3. The circuit of claim 1 wherein the comparator compares an output of the first register with an output of the counter, an output of the comparator comprising the signals, the signals comprising pulses modulated as a function of the depth.

4. The circuit of claim 1 wherein the second adder is triggered by an output of the comparator to add a previous relative timing with a scale value, a sum from the second adder comprising the relative timing.

5. The circuit of claim 1 wherein the sub-array beamformer comprises a phase rotator, the phase rotator operable to rotate a phase of the ultrasound signals by the relative timing.

6. The circuit of claim 1 wherein the first register is configured to be loaded with a constant, the constant depending on a position of the element relative to a scan line.

7. The circuit of claim 1 wherein the first register is configured to be loaded with an initial value depending on a position of the element relative to a scan line and to replace the initial value for increasing depths.

8. The circuit of claim 1 wherein the relative timing is a log(x), 1/x, or arctan(x), where x is depth.

9. The circuit of claim 1 wherein the signals generated by the comparator comprise the reciprocal of a derivative, the derivative comprising the second order polynomial.

10. The circuit of claim 1 wherein the relative timing for the element in the sub-array is generated without a multiplier, without a divider, and without a cordic operation.

11. The circuit of claim 1 further comprising second, third, and fourth registers, the first, second, third, and fourth registers loaded with initial values by scan line, the initial values corresponding to a curve fit of Euclidian distance to the circuit operation.

12. A method for dynamic phase or delay calculation in a sub-array of ultrasound imaging, the method comprising:
determining dynamic timing relationships between elements of a sub-array with a log(x), arctan(x), or 1/x function, where x is time; and
beamforming signals from the elements of the sub-array as a function of the dynamic timing relationships.

13. The method of claim 12 wherein determining comprises determining the dynamic timing relationships as relative phases as a function of depth.

14. The method of claim 12 wherein determining comprises determining the dynamic timing relationships as relative delays as a function of depth.

15. The method of claim 12 wherein beamforming comprises:
relatively delaying or phasing signals from the elements as a function of the dynamic timing relationships; and
summing the relatively delayed or phased signals from the elements.

16. The method of claim 12 wherein determining comprises producing, with a comparator, a reciprocal of a derivative.

17. The method of claim 12 wherein determining comprises:
generating a second order polynomial as a function of the time;
calculating a reciprocal of the second order polynomial; and
integrating the reciprocal;
wherein a result of the integrating comprises a dynamic timing relationship of one of the elements.

18. The method of claim 12 wherein determining comprises:
first adding a first value with a second value for each of multiple increments in the time, a result of the first adding comprising the second value for a next increment;
second adding the second value with a third value for each of the multiple increments in the time, a result of the second adding comprising the third value for the next increment;
comparing the third value with a counter;
third adding a scale value to a previous dynamic timing relationship when triggered by an output of the comparing, a result of the third adding comprising a dynamic timing relationship for one of the elements.

19. A circuit for dynamic operation in a sub-array of ultrasound imaging, the circuit comprising:
a comparator to produce a reciprocal of a derivative;
a sub-array beamformer to delay or phase ultrasound signals for an element based on the reciprocal of the derivative.

20. The circuit of claim 19 further comprising:
first adders and registers to generate the derivative as a function of depth;
a counter connected with the comparator, the comparator operable to produce the reciprocal by comparison of an output of the counter and the derivative; and
a second adder to generate a relative timing for the element from the reciprocal, the relative timing being the delay or phase as a function of depth.
